(19) United States
(12) Patent Application Publication
(10) Pub. No.: US 2018/0078489 A1
(43) Pub. Date: Mar. 22, 2018

(54) SOLID ANHYDROUS COMPOSITION COMPRISING A COMBINATION OF WAXES

(71) Applicant: L'OREAL, Paris (FR)

(72) Inventor: Zhen XU, Shanghai (CN)

(73) Assignee: L'OREAL, Paris (FR)

(21) Appl. No.: 15/567,438

(22) PCT Filed: Jul. 14, 2015
(86) PCT No.: PCT/CN2015/083964

§ 371 Date: Oct. 18, 2017

(51) Int. Cl. A61K 8/92; A61Q 1/06; A61K 8/37; A61K 8/31; A61K 8/02; A61K 8/81; A61K 8/34

(52) U.S. Cl. CPC A61K 8/925; A61Q 1/06; A61K 8/37; A61K 8/92; A61K 2800/5922; A61K 8/0229; A61K 8/8111; A61K 8/342; A61K 2800/43; A61K 8/31

(57) ABSTRACT

Disclosed is, in general, a solid anhydrous composition for application onto a targeted substrate. More particularly, disclosed is a non-liquid anhydrous composition with a combination of waxes, comprising a) at least one wax with a melting point greater than or equal to 60° C.; b) at least one wax, different from a), of solid linear ester derived from C6-C30 fatty acid, c) at least one nonvolatile oil, and d) at least one pasty compound, wherein the weight ratio of a) and b) is from 1 to 4.

### TECHNICAL FIELD

The present invention relates, in general, to a solid, anhydrous composition for application onto a targeted substrate. More particularly, the present invention relates to a solid anhydrous composition which is cast cosmetic composition.

### BACKGROUND OF THE INVENTION

Compositions for caring for and/or making up the skin and/or the lips generally contain one or more fatty substances and are structured with a “structuring” or “gelling” agent, conventionally a wax or a polymer, to improve the stiffness of the compositions and especially to obtain solid compositions, preferably in the form of sticks. These solid compositions remain stable and in particular do not exude, especially at room temperature.

Needless to say, the galenical form of these compositions must, on the one hand, satisfy mechanical requirements in order to ensure the glidance and wear properties of the stick during application and to prevent it from breaking, and, on the other hand, satisfy transfer qualities so as to ensure comfortable application and also a sufficient and good-quality deposit on the skin or the lips. Efforts have been made to this end. Waxes with a melting point of greater than or equal to 60° C. are known to be used for formulating solid anhydrous compositions with sufficient solidity so as to prevent it from breaking. For example, it is known to use polyethylene and hydrogenated myristyl olive esters in combination, to formulate a lip stick which does not break when applying on the skin or lips at high temperature, such as 37° C.

However the compositions as such are found not satisfying, especially in terms of aesthetic appearance after a long period of storage. It is observed that white crystals appear on the surface of the final product, which is a solid cast product of the composition, after 3 months storage.

In order to solve the issue, other waxes with different melting points, such as beeswax, are used. However it is observed that the sensory and spreading of the compositions are not satisfying. The final product is draggy during application, or occurs to be too oily after application on the skin or the lips.

Therefore there exists a need for a composition which provides an improved aesthetic appearance without white crystal on the surface after long time storage, improved sensory after application, in particular no oily feeling, and an improved spreading, by which reducing or diminishing the draggy feeling during application, while retaining sufficient mechanical property to avoid breaking while application at high temperatures, such as 37° C. or above.

### DISCLOSURE OF INVENTION

The inventors found that such needs can be achieved by a combination of waxes with specific weight ratio between one another.

The invention is thus relating to a solid anhydrous composition with a combination of waxes, comprising:
a) at least one wax with a melting point greater than or equal to 60° C.;
b) at least one wax, different from a), of solid linear ester derived from C₆-C₃₀ fatty acid,
c) at least one nonvolatile oil, and
d) at least one pasty compound,
wherein the weight ratio of a) and b) is from 0.05 to 4.

Another object of the invention is a non-therapeutic method for treating a keratin material, preferably the skin and/or the lips, comprising the step of applying to the keratin material the composition as described above.

The term “solid” composition means a composition which the hardness at 20° C. and at atmospheric pressure (760 mmHg) is greater than or equal to 30 Nm⁻¹ when it is measured according to the protocol described below.

The composition whose hardness is to be determined is stored at 20° C. for 24 hours before measuring.

The hardness may be measured at 20° C. via the “cheese wire” method, which consists in transversely cutting a wand of product, which is preferably a circular cylinder, by means of a rigid tungsten wire 250 μm in diameter, by moving the wire relative to the stick at a speed of 100 mm/minute.

The hardness of the compositions of the invention, expressed in Nm⁻¹, is measured using a DFGS2 tensile testing machine from the company Indelco-Chatillon.

The measurement is repeated three times and then averaged. The average of the three values read using the tensile testing machine mentioned above, noted Y, is given in grams. This average is converted into Newtons and then divided by L which represents the longest distance through which the wire passes. In the case of a cylindrical wand, L is equal to the diameter (in metres).

The hardness is converted into Nm⁻¹ by the equation below:

(Y×10⁻³×9.8)/L

For a measurement at a different temperature, the stick is stored for 24 hours at this new temperature before the measurement.

According to this measuring method, the composition according to the invention preferably has a hardness at 20° C. and at atmospheric pressure of greater than or equal to 40 Nm⁻¹ and preferably greater than 50 Nm⁻¹.

Preferably, the composition according to the invention especially has a hardness at 20° C. of less than 500 Nm⁻¹, especially less than 400 Nm⁻¹ and preferably less than 300 Nm⁻¹.

Advantageously, these compositions have a shear value ranging from 50 to 120 and preferably from 70 to 100 gF. Thus, these compositions may be formulated in standard packaging that does not require any composition support means.

For the purposes of the invention, the term “anhydrous composition” means a composition containing less than 2% and preferably less than 0.5% by weight of water relative to the total weight of the composition. Where appropriate, such small amounts of water may be provided by ingredients of the composition that contain it in residual amount, but are not deliberately provided.

Preferably, the “keratin material” according to the present invention is the skin and the lips. By “skin”, we intend to mean all the body skin, including the scalp. Still preferably, the keratin material is the lips.

### DETAILED DESCRIPTION OF THE INVENTION

### Waxes a)

According to the present invention, the composition comprises at least one wax with a melting point greater than or equal to 60° C.

The wax under consideration in the context of the present invention is generally a lipophilic compound that is solid at room temperature (25° C.), with a solid/liquid reversible change of state, having a melting point of greater than or equal to 60° C., which may be up to 200° C. and in particular up to 120° C.

The wax a) according to the present invention may be of natural origin, for instance plant origin, animal origin, or mineral origin. It may also be of synthetic origin, or a mixture thereof.

As illustrations of waxes that are suitable for the invention, mention may be made especially of:

waxes of natural origin, for instance beeswax, lanolin wax, Chinese insect waxes, rice bran wax, carnauba wax, candelilla wax, ouricury wax, esparto grass wax, berry wax, shellac wax, Japan wax and sumach wax, montan wax, orange wax, lemon wax, ozokerite, and hydrogenated oils such as hydrogenated jojoba oil;

waxes of synthetic origin, such as microcrystalline waxes, paraffin wax, polyethylene waxes, which is derived from the polymerization of ethylene; the waxes obtained by Fischer-Tropsch synthesis (Fischer-Tropsch waxes) and waxy copolymers, and also esters thereof; fatty acids or esters obtained by catalytic hydrogenation of animal or plant oils containing linear or branched C₈-C₃₂ fatty chains, preferably C₁₆ to C₁₈ chains; silicone waxes; fluoro waxes; or a mixture thereof.

Preferably the wax a) of the present invention is selected from those of synthetic origin.

Mentions may be made of polyethylene waxes which, for example, those sold under the tradename Performalene 500-L Polyethylene by the company New Phase Technologies.

Examples of waxes obtained by Fischer-Tropsch synthesis, or Fischer-Tropsch waxes can be mentioned are those synthetic wax sold under the tradename Cirebelle 108 by the company Cirebelle (Technical name: Paraffin wax), for example.

Mention may also be made of fatty acids obtained by catalytic hydrogenation of animal or plant oils containing linear or branched C₈-C₃₂ fatty chains, preferably C₁₆ to C₁₈ chains. Among these compounds, mention may be made especially of stearic acid, palmitic acid, or a mixture thereof. The fatty acids used in the current invention are commercially available under the trade names, for example, AEC Stearic Acid sold by A & E Connock (Perfumery & Cosmetics) Ltd., Emersol sold by Emery Oleochemical LLC, Palmitic Acid PC sold by Protameen Chemicals, Inc.

Mentions may also be made of this type of waxes, for example those obtained by hydrogenation of castor oil esterified with cetyl alcohol, sold under the names Phytowax ricin 16L64® and 22L73® by the company Sophim, may also be used. Such waxes are described in patent application FR-A-2 792 190.

Mentions may be made of waxes obtained by hydrogenation of olive oil esterified with C₁₂ to C₁₈ chain fatty alcohols such as those sold by the company SOPHIM under the brand names Phytowax Olive 12L44, 14L48, 16L55 and 18L57.

Mention may also be made of silicone waxes such as C₃₀-₄₅ alkyl dimethicone; and fluoro waxes.

Preferably, the wax a) of the present invention is selected from polyethylene waxes.

Advantageously, a composition of the invention may comprise from 1% to 20% by weight and preferably from 2% to 15% by weight and more preferably from 5% to 10% by weight of wax(es) a), relative to the total weight of the said composition.

### Waxes b)

The composition of the present invention comprises at least one wax of solid linear ester derived from C₆-C₃₀ fatty acid, which is different from the wax a) as described above.

Examples of the fatty acids mentioned above are caprylic acid, capric acid, lauric acid, myristic acid, palmitic acid, stearic acid, arachidic acid, behenic acid, lignoceric acid, and cerotic acid, saturated or unsaturated.

For the purposes of the invention, the solid linear esters contain in total from 25 to 36 carbon atoms and have melting points of between 38° C. and 60° C. Preferably, a linear ester is said to be in the solid state when all of its mass is in solid crystalline form at room temperature.

The solid linear esters that are suitable for use in the invention are preferably chosen from the group comprising stearyl stearate, tetradecyl tetradecanoate (INCI name: myristyl myristate), cetyl myristate, stearyl myristate, myristyl palmitate, stearyl palmitate, myristyl stearate, cetyl stearate, stearyl stearate and cetyl palmitate, and mixtures thereof.

As stearyl stearate that is most particularly suitable for use in the invention, use may be made, for example, of the product sold under the name Liponate® SS sold by the company Lipo Chemicals.

According to a preferred embodiment, the wax b) according to the invention is solid linear ester derived from C₁₀-C₂₂ fatty acid, and containing from 25 to 36 carbon atoms.

More preferably, the wax b) according to the invention is solid linear ester derived from C₁₂-C₂₀ fatty acid, and containing from 28 to 36 carbon atoms. As examples of mixtures of solid linear esters that are most particularly suitable for the invention, mention may be made of myristyl/cetyl/stearyl myristate/palmitate/stearate (INCI name: cetyl esters (and) cetyl esters), for example sold under the names Crocamol® MS-PA-(MH), Crocamol® MS-PA-(BR), Crocamol® MS-PA-(SG) and Crocamol®MS-PA-(MV) sold by the company Croda, Miraceti® sold by the company Laserson, and Paester 9161 sold by the company Patech Fine Chemicals.

Preferably, the wax b) of the present invention is selected from myristyl stearate, myristyl palmitate, or a mixture thereof.

According to one preferred embodiment, the composition according to the invention comprises a content of solid linear or ester(s) ranging from 0.5% to 20% by weight, preferably from 1% to 10% by weight, relative to the total weight of the composition.

### Weight Ratio of the Waxes a) and b)

According to the present invention, the weight ratio of the wax a) and b) is from 1 to 4.

Preferably, the weight ratio of the wax a) and b) is from 0.5 to 2.

The weight ratio of the wax a) and b) provides to the composition of the invention a desired mechanical property so as to prevent it from breaking when applying to the skin or the lips at high temperature, such as 37° C., so as to provide a desired spreading without draggy feeling during application, as well as a desired sensory without oily feelings as well as a good pay-off effect after application. Moreover, it provides the composition with an improved aesthetic appearance after a long time storage, such as 3 months.

### Nonvolatile Oils

The composition of the present invention comprises at least one nonvolatile oil.

By “oil” it differs from the waxes as described above, in that the oils are liquid at room temperature (25° C.) and atmospheric pressure (1.013.10⁵ Pa or 760 mmHg).

The term “nonvolatile oil” means an oil that remains on keratin materials, at room temperature and atmospheric pressure, for at least several hours and that especially has a vapor pressure of less than 10⁻³ mmHg (0.13 Pa).

A nonvolatile oil may also be defined as having an evaporation rate such that, under the conditions defined previously, the amount evaporated after 30 minutes is less than 0.07 mg/cm².

The oil may be chosen from polar oils, apolar oils, or mixtures thereof.

### Polar Oils

For the purposes of the present invention, the term “polar oil” means an oil whose solubility parameter at 25° C., δa, is other than 0 (J/cm³)^{1/2}.

The composition according to the invention may comprise at least one polar nonvolatile oil, chosen from hydrocarbon-based oils, fluoro oils, silicone oils, or a mixture thereof. The term “silicone oil” means an oil containing at least one silicon atom, and especially containing Si—O groups.

The term “fluoro oil” means an oil containing at least one fluorine atom.

These oils may be of vegetable, mineral or synthetic origin.

The term “hydrocarbon-based oil” means an oil formed essentially from, or even constituted by, carbon and hydrogen atoms, and optionally oxygen and nitrogen atoms, and not containing any silicon or fluorine atoms. It may contain alcohol, ester, ether, carboxylic acid, amine and/or amide groups.

According to one preferred mode, the composition comprises nonvolatile hydrocarbon-based polar oil(s).

The nonvolatile hydrocarbon-based polar oil may be chosen from the list of oils below, and mixtures thereof:

C₁₀-C₂₆ alcohols, preferably monoalcohols;

More particularly, the C₁₀-C₂₆ alcohols are saturated or unsaturated, and branched or unbranched, and comprise from 10 to 26 carbon atoms.

Preferably, the C₁₀-C₂₆ alcohols are fatty alcohols, which are preferably branched when they comprise at least 16 carbon atoms.

As examples of fatty alcohols that may be used according to the invention, mention may be made of linear or branched fatty alcohols, of synthetic origin or alternatively of natural origin, for example alcohols derived from plant material (coconut, palm kernel, palm, etc.) or animal material (tallow, etc.).

Needless to say, other long-chain alcohols may also be used, for instance ether alcohols or alternatively “Guerbet” alcohols.

Finally, use may also be made of certain more or less long fractions of alcohols of natural origin, for instance coconut (C₁₂ to C₁₆) or tallow (C₁₆ to C₁₈) or compounds of diol or cholesterol type.

Use is preferably made of a fatty alcohol comprising from 10 to 24 carbon atoms and more preferentially from 12 to 22 carbon atoms.

As particular examples of fatty alcohols that may preferably be used, mention may be made especially of lauryl alcohol, isostearyl alcohol, oleyl alcohol, 2-butyloctanol, 2-undecylpentadecanol, 2-hexyldecyl alcohol, isocetyl alcohol and octyldodecanol, and mixtures thereof.

According to one advantageous embodiment of the invention, the alcohol is chosen from octyldodecanol, such as the product sold under the tradename Eutanol G® by the company BASF.

optionally hydroxylated monoesters, diesters or triesters of a C₂-C₈ monocarboxylic or polycarboxylic acid and of a C₂-C₈ alcohol.

In particular:
optionally hydroxylated monoesters of a C₂-C₈ carboxylic acid and of a C₂-C₈ alcohol,
optionally hydroxylated diesters of a C₂-C₈ dicarboxylic acid and of a C₂-C₈ alcohol, such as diisopropyl adipate, 2-diethylhexyl adipate, dibutyl adipate, diisostearyl adipate or 2-diethylhexyl succinate,
optionally hydroxylated triesters of a C₂-C₈ tricarboxylic acid and of a C₂-C₈ alcohol, such as citric acid esters, such as trioctyl citrate, triethyl citrate, acetyl tributyl citrate or tributyl citrate,
   esters of a C₂-C₈ polyol and of one or more C₂-C₈ carboxylic acids, such as glycol diesters of monoacids, such as neopentyl glycol diheptanoate, or glycol triesters of monoacids, such as triacetin.
   ester oils, in particular containing at least 18 carbon atoms and even more particularly between 18 and 70 carbon atoms.

Examples that may be mentioned include monoesters, diesters or triesters.

The ester oils may be hydroxylated or non-hydroxylated.

The non-volatile ester oil may for example be chosen from:
monoesters comprising at least 18 carbon atoms and even more particularly containing between 18 and 40 carbon atoms in total, in particular the monoesters of formula R₁COOR₂ in which R₁ represents a saturated or unsaturated, linear or branched or aromatic fatty acid residue comprising from 4 to 40 carbon atoms and R₂ represents a hydrocarbon-based chain, which is in particular branched, containing from 4 to 40 carbon atoms, with the proviso that the sum of the carbon atoms of the radicals R₁ and R₂ is greater than or equal to 18, for instance Purcellin oil (cetostearyl octanoate), isononyl isononanoate, C₁₂ to C₁₅ alkyl benzoates, 2-ethylhexyl palmitate, octyldodecyl neopentanoate, 2-octyldodecyl stearate, 2-octyldodecyl erucate, isostearyl isostearate, C₁₂-C₁₅ alkyl benzoates, such as 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate or 2-octyldodecyl myristate.

Preferably, they are esters of formula R₁COOR₂ in which R₁ represents a linear or branched fatty acid residue comprising from 4 to 40 carbon atoms and R₂ represents a hydrocarbon-based chain that is in particular branched, containing from 4 to 40 carbon atoms, R₁ and R₂ being such that the sum of the carbon atoms of the radicals R₁ and R₂ is greater than or equal to 18.

Even more particularly, the ester comprises between 18 and 40 carbon atoms in total.

Preferred monoesters that may be mentioned include isononyl isononanoate, oleyl erucate and/or 2-octyldodecyl neopentanoate;
monoesters of a fatty acid, in particular containing at least 18 carbon atoms and even more particularly from 18 to 22 carbon atoms, and especially of lanolic acid, oleic acid, lauric acid or stearic acid, and of diols, for instance propylene glycol monoisostearate.
diesters especially containing at least 18 carbon atoms and even more particularly comprising between 18 and 60 carbon atoms in total and in particular between 18 and 50 carbon atoms in total. Use may be made especially of diesters of a dicarboxylic acid and of monoalcohols, preferably such as diisostearyl malate, or glycol diesters of monocarboxylic acids, such as neopentyl glycol diheptanoate, propylene glycol dioctanoate, diethylene glycol diisononanoate or polyglyceryl-2 diisostearate (in particular such as the compound sold under the commercial reference Dermol DGDIS by the company Akzo);
hydroxylated monoesters and diesters, preferably with a total carbon number of at least 18 carbon atoms and even more particularly ranging from 18 to 70, for instance polyglyceryl-3 diisostearate, isostearyl lactate, octyl hydroxystearate, octyldodecyl hydroxystearate, diisostearyl malate or glyceryl stearate;
triesters especially containing at least 35 carbon atoms and even more particularly comprising between 35 and 70 carbon atoms in total, in particular such as triesters of a tricarboxylic acid, such as triisostearyl citrate, or tridecyl trimellitate, or glycol triesters of monocarboxylic acids such as polyglyceryl-2 triisostearate;
tetraesters especially containing at least 35 carbon atoms and even more particularly with a total carbon number ranging from 35 to 70, such as pentaerythritol or polyglycerol tetraesters of a monocarboxylic acid, for instance pentaerythrityl tetrapelargonate, pentaerythrityl tetraisostearate, pentaerythrityl tetraisononanoate, glyceryl tris(2-decyl)tetradecanoate, polyglyceryl-2 tetraisostearate or pentaerythrityl tetrakis(2-decyl)tetradecanoate;
polyesters obtained by condensation of an unsaturated fatty acid dimer and/or trimer and of diol, such as those described in patent application FR 0 853 634, in particular such as dilinoleic acid and 1,4-butanediol. Mention may especially be made in this respect of the polymer sold by Biosynthis under the name Viscoplast 14436H (INCI name: dilinoleic acid/butanediol copolymer), or else copolymers of polyols and of dimer diacids, and esters thereof, such as Hailucent ISDA;
esters and polyesters of diol dimer and of monocarboxylic or dicarboxylic acid, such as esters of diol dimer and of fatty acid and esters of diol dimer and of dicarboxylic acid dimer, in particular which may be obtained from a dicarboxylic acid dimer derived in particular from the dimerization of an unsaturated fatty acid especially of C₈ to C_{34,} especially of C₁₂ to C_{22,} in particular of C₁₈ to C₂₀ and more particularly of C₁₈, such as esters of dilinoleic diacids and of dilinoleic diol dimers, for instance those sold by the company Nippon Fine Chemical under the trade names Lusplan DD-DA5® and DD-DA7®;
polyesters resulting from the esterification of at least one triglyceride of hydroxylated carboxylic acid(s) with an aliphatic monocarboxylic acid and with an aliphatic dicarboxylic acid, which is optionally unsaturated, for instance the succinic acid and isostearic acid castor oil sold under the reference Zenigloss by Zenitech;
hydrocarbon-based plant oils such as fatty acid triglycerides (which are liquid at room temperature), especially of fatty acids containing at least 7 carbon atoms and even more particularly containing from 7 to 40 carbon atoms, such as heptanoic or octanoic acid triglycerides or jojoba oil; mention may be made in particular of saturated triglycerides such as caprylic/capric triglycerides and mixtures thereof, for example such as the product sold under the reference Myritol 318 from Cognis, glyceryl triheptanoate, glyceryl trioctanoate, and C₁₈₋₃₆ acid triglycerides such as those sold under the reference Dub TGI 24 by Stearineries Dubois, and unsaturated triglycerides such as castor oil, olive oil, ximenia oil or pracaxi oil;
   vinylpyrrolidone/1-hexadecene copolymers, for instance the product sold under the name Antaron V-216 (also known as Ganex V216) by the company ISP (MW=7300 g/mol);
   C₁₂-C₂₆ fatty acids, preferably C₁₂-C₂₂ fatty acids, which are preferably unsaturated, such as oleic acid, linoleic acid or linolenic acid, and mixtures thereof;
   dialkyl carbonates, the 2 alkyl chains possibly being identical or different, such as dicaprylyl carbonate sold under the name Cetiol CC® by Cognis;
   or mixtures thereof.

Preferably, the hydrocarbon-based polar oil according to the invention is selected from the group consisting of C₁₀-C₂₆ alcohols, preferably monoalcohols, ester oils, in particular containing at least 18 carbon atoms and even more particularly between 18 and 70 carbon atoms, or a mixture thereof.

More preferably, the hydrocarbon-based polar oil of the present invention is selected from the group consisting of C₁₂-C₂₂ alcohols, diesters especially containing at least 18 carbon atoms and even more particularly comprising between 18 and 60 carbon atoms in total, or a mixture thereof.

Even more preferably, the nonvolatile hydrocarbon-based polar oil is selected from the group consisting of diisostearyl malate (for instance the compound sold by the company Lubrizol under the trade name Schercemol Dism Ester), octyldodecanol (for instance Eutanol® G from the ABSF company), or a mixture thereof.

### Apolar Oils

For the purposes of the present invention, the term “apolar oil” means an oil whose solubility parameter at 25° C., δa, is equal to 0 (J/cm³)^{1/2}.

The definition and calculation of the solubility parameters in the Hansen three-dimensional solubility space are described in the article by C. M. Hansen: “The three dimensional solubility parameters”, J. Paint Technol. 39, 105 (1967).

According to this Hansen space:
δD characterizes the London dispersion forces derived from the formation of dipoles induced during molecular impacts;
δp characterizes the Debye interaction forces between permanent dipoles and also the Keesom interaction forces between induced dipoles and permanent dipoles;
δh characterizes the specific interaction forces (such as hydrogen bonding, acid/base, donor/acceptor, etc.); and
δa is determined by the equation: δa=(δp²+δh²)^{1/2}.

The parameters δp, δh, δD and δa are expressed in (J/cm³)^{1/2}.

As the nonvolatile apolar hydrocarbon-based oil is apolar, the compound is then free of oxygen, nitrogen atom(s).

More preferably the nonvolatile apolar oil is nonvolatile hydrocarbon-based apolar oil.

Preferably, the nonvolatile hydrocarbon-based apolar oil may be chosen from linear or branched hydrocarbons of mineral or synthetic origin, such as:
liquid paraffin or derivatives thereof,
squalane,
isoeicosane,
liquid petroleum jelly,
naphthalene oil,
polybutenes such as Indopol H-100 (molar mass or MW=965 g/mol), Indopol H-300 (MW=1340 g/mol) and Indopol H-1500 (MW=2160g/mol) sold or manufactured by the company Ineos,
hydrogenated polyisobutylenes such as Parleam® sold by the company Nippon Oil Fats, Panalane H-300 E sold or manufactured by the company Amoco (MW=1340 g/mol), Viseal 20000 sold or manufactured by the company Synteal (MW=6000 g/mol) and Rewopal PIB 1000 sold or manufactured by the company Witco (MW=1000 g/mol),
decene/butene copolymers, polybutene/polyisobutene copolymers, especially Indopol L-14,
polydecenes and hydrogenated polydecenes such as: Puresyn 10 (MW=723 g/mol) and Puresyn 150 (MW=9200 g/mol) sold or manufactured by the company Mobil Chemicals,
and mixtures thereof.

Preferably, the nonvolatile oil of the present invention is polybutene. Preferably, the composition of the invention comprises at least one nonvolatile oil selected from the group consisting of nonvolatile hydrocarbon-based polar oils, hydrocarbon-based apolar oils, or a mixture thereof.

More preferably, the composition of the invention comprises at least one nonvolatile oil selected from the group consisting of hydrocarbon-based mono- or di-esters of the carboxylic acid residue containing from 2 to 30 carbon atoms, and the alcohol residue represents a hydrocarbon-based chain containing from 1 to 30 carbon atoms, fatty alcohols containing from 12 to 36 carbon atoms, saturated or unsaturated, linear or branched, polybutenes, or a mixture thereof.

Even more preferably, the nonvolatile oil is selected from the group consisting of diisostearyl malate, octyldodecanol, polybutene, or a mixture thereof.

Preferably, the nonvolatile oil is present in an amount ranging from 3% to 80% by weight, preferably from 5% to 60% by weight, relative to the total weight of the composition.

### Volatile Oils

Although this does not represent a preference of the present invention, the composition may optionally comprise at least one volatile oil as additional oil.

Such a volatile oil may especially be a hydrocarbon-based oil or a silicone oil.

For the purposes of the invention, the term “volatile oil” means an oil that is capable of evaporating on contact with keratin materials in less than one hour, at room temperature and atmospheric pressure (760 mmHg).

The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

These oils may be hydrocarbon-based oils, silicone oils or fluoro oils, or mixtures thereof. In particular, volatile oils that may be mentioned include volatile hydrocarbon-based oils and especially volatile hydrocarbon-based oils with a flash point of less than or equal to 80° C. (the flash point is in particular measured according to ISO Standard 3679), such as hydrocarbon-based oils containing from 8 to 14 carbon atoms, and especially:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane, and, for example, the oils sold under the trade name Isopar or Permethyl,
linear alkanes, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C₁₃) obtained in Examples 1 and 2 of patent application WO 2008/155 059 from the company Cognis, and mixtures thereof.

As other volatile hydrocarbon-based oils, and especially as volatile hydrocarbon-based oils with a flash point of less than or equal to 80° C., mention may also be made of ketones that are liquid at room temperature, such as methyl ethyl ketone or acetone; short-chain esters (containing from 3 to 8 carbon atoms in total) such as ethyl acetate, methyl acetate, propyl acetate or n-butyl acetate; ethers that are liquid at room temperature, such as diethyl ether, dimethyl ether or dichlorodiethyl ether; alcohols and especially linear or branched lower monoalcohols containing from 2 to 5 carbon atoms, such as ethanol, isopropanol or n-propanol.

Advantageously, the composition comprises less than 5% and better still less than 2% by weight of volatile oil relative to the total weight of the composition.

Preferably, the composition is free of volatile oil.

### Pasty Compounds

The composition of the present invention comprises at least one pasty compound.

The term “pasty compound” within the meaning of the present invention is understood to mean a lipophilic fatty compound with a reversible solid/liquid change in state which exhibits, in the solid state, an anisotropic crystalline arrangement and which comprises, at a temperature of 23° C., a liquid fraction and a solid fraction.

In other words, the starting melting point of the pasty fatty substance can be less than 23° C.

The liquid fraction of the pasty compounds, measured at 23° C., can represent from 9 to 97% by weight of the pasty compounds. At 23° C., this liquid fraction preferably represents between 15 and 85% by weight, more preferably between 40 and 85% by weight.

Within the meaning of the invention, the melting point corresponds to the temperature of the most endothermic peak observed by thermal analysis (DSC) as described in Standard ISO 11357-3: 1999. The melting point of a pasty compounds can be measured using a differential scanning calorimeter (DSC), for example the calorimeter sold under the name “MDSC 2920” by TA Instruments.

The measurement protocol is as follows:

A 5 mg sample of pasty compounds placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of pasty compounds is measured as a function of the temperature. The melting point of the pasty compounds is the value of the temperature corresponding to the tip of the peak of the curve representing the variation in the difference in power absorbed as a function of the temperature.

The liquid fraction by weight of the pasty compounds at 23° C. is equal to the ratio of the enthalpy of fusion consumed at 23° C. to the enthalpy of fusion of the pasty fatty substance.

The enthalpy of fusion of the pasty fatty substance is the enthalpy consumed by the latter to change from the solid state to the liquid state. The pasty fatty substance is “in the solid state” when the whole of its mass is in the solid crystalline form. The pasty compounds is “in the liquid state” when the whole of its mass is in the liquid form.

The enthalpy of fusion of the pasty compounds is equal to the area under the curve of the thermogram obtained using a differential scanning calorimeter (DSC), such as the calorimeter sold under the name MDSC 2920 by TA Instruments, with a rise in temperature of 5 or 10° C. per minute, according to Standard ISO 11357-3:1999.

The enthalpy of fusion of the pasty compounds is the amount of energy necessary to change the pasty compounds from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compounds measured at 32° C. preferably represents from 30 to 100% by weight of the pasty compounds, preferably from 50 to 100% by weight of the pasty compounds, more preferably from 60 to 100% by weight of the pasty compounds. When the liquid fraction of the pasty compounds measured at 32° C. is equal to 100%, the temperature of the end of the melting range of the pasty compounds is less than or equal to 32° C.

The liquid fraction of the pasty compounds measured at 32° C. is equal to the ratio of the enthalpy of fusion consumed at 32° C. to the enthalpy of fusion of the pasty compounds. The enthalpy of fusion consumed at 32° C. is calculated in the same way as the enthalpy of fusion consumed at 23° C.

The pasty compound is advantageously chosen from the following compounds, alone or in combination:
lanolin and derivatives thereof;
petroleum jelly, in particular the product whose INCI name is petrolatum and which is sold under the name Ultima White PET USP by the company Calumet Specialty,
polyol ethers chosen from ethers of pentaerythritol and of polyalkylene glycol, ethers of fatty alcohol and of sugar, and mixtures thereof, the ethers of pentaerythritol and of polyethylene glycol comprising 5 oxyethylene units (5 OE) (CTFA name: PEG-5 Pentaerythrityl Ether), polypropylene glycol pentaerythrityl ether comprising five oxypropylene (5 OP) units (CTFA name: PEG-5 Pentaerythrityl Ether) and mixtures thereof, and more especially the mixture PEG-5 Pentaerythrityl Ether, PPG-5 Pentaerythrityl Ether and soybean oil, sold under the name Lanolide by the company, Vevy, which is a mixture in which the constituents are in a 46/46/8 weight ratio: 46% PEG-5 Pentaerythrityl Ether, 46% PPG-5 Pentaerythrityl Ether and 8% soybean oil;
polymeric or nonpolymeric silicone compounds,
polymeric or nonpolymeric fluoro compounds,
vinyl polymers, especially:
   olefin homopolymers and copolymers,
   hydrogenated diene homopolymers and copolymers,
   linear or branched oligomers, homopolymers and copolymers of alkyl (meth)acrylates preferably containing a C₈-C₃₀ alkyl group,
   oligomers, homopolymers and copolymers of vinyl esters containing C₈-C₃₀ alkyl groups, such as vinyl ester homopolymers containing C₈-C₃₀ alkyl groups, such as polyvinyl laurate (sold especially under the reference Mexomer PP by the company Chimex) and arachidyl propionate sold under the brand name Waxenol 801 by Alzo;
   oligomers, homopolymers and copolymers of vinyl ethers containing C₈-C₃₀ alkyl groups;
liposoluble polyethers resulting from the polyetherification between one or more C₂-C₁₀₀ and preferably C₂-C₅₀ diols,

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will be made especially of copolymers such that the long-chain alkylene oxides are arranged in blocks having an average molecular weight from 1000 to 10 000, for example a polyoxyethylene/polydodecyl glycol block copolymer such as the ethers of dodecanediol (22 mol) and of polyethylene glycol (45 OE) sold under the brand name Elfacos ST9 by Akzo Nobel.
esters especially those chosen from:
   esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, preferably such as bis-diglyceryl polyacyladipate-2 (INCI name) sold under the brand name Softisan 649 by the company Cremer Oleo,
   vinyl ester homopolymers containing C₈-C₃₀ alkyl groups, such as polyvinyl laurate (sold especially under the reference Mexomer PP by the company Chimex) and arachidyl propionate sold under the brand name Waxenol 801 by Alzo,
   phytosterol esters,
   fatty acid triglycerides and derivatives thereof, for instance triglycerides of fatty acids, which are especially C₁₀-C_{18,} and partially or totally hydrogenated such as those sold under the reference Softisan 100 by the company Sasol,
   pentaerythritol esters,
   noncrosslinked polyesters resulting from polycondensation between a linear or branched C₄-C₅₀ dicarboxylic acid or polycarboxylic acid and a C₂-C₅₀ diol or polyol,
   aliphatic esters of an ester, resulting from the esterification of an aliphatic hydroxycarboxylic acid ester with an aliphatic carboxylic acid. Preferably, the aliphatic carboxylic acid comprises from 4 to 30 and preferably from 8 to 30 carbon atoms. It is preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid and docosanoic acid, and mixtures thereof. The aliphatic carboxylic acid is preferably branched. The aliphatic hydroxycarboxylic acid ester is advantageously derived from a hydroxylated aliphatic carboxylic acid containing from 2 to 40 carbon atoms, preferably from 10 to 34 carbon atoms and better still from 12 to 28 carbon atoms, and from 1 to 20 hydroxyl groups, preferably from 1 to 10 hydroxyl groups and better still from 1 to 6 hydroxyl groups. The aliphatic hydroxycarboxylic acid ester is chosen from:
   partial or total esters of saturated linear mono-hydroxylated aliphatic monocarboxylic acids;
   partial or total esters of unsaturated monohydroxylated aliphatic monocarboxylic acids;
   partial or total esters of saturated monohydroxylated aliphatic polycarboxylic acids;
   partial or total esters of saturated polyhydroxylated aliphatic polycarboxylic acids;
   partial or total esters of C₂ to C₁₆ aliphatic polyols that have reacted with a monohydroxylated or polyhydroxylated aliphatic monocarboxylic or polycarboxylic acid,
   and mixtures thereof;
   esters of a diol dimer and of a diacid dimer, where appropriate esterified on their free alcohol or acid functional group(s) with acid or alcohol radicals, especially dimer dilinoleate esters; such esters may be chosen especially from the esters having the following INCI nomenclature: Bis-Behenyl/Isostearyl/Phytosteryl Dimer Dilinoleyl Dimer Dilinoleate (Plandool G), Phytosteryl/Isostearyl/Cetyl/Stearyl/Behenyl Dimer Dilinoleate (Plandool H or Plandool S), and mixtures thereof,
   hydrogenated plant oils such as soybean oil, hydrogenated coconut oil, hydrogenated rape seed oil, mixtures of hydrogenated plant oils such as the mixture of hydrogenated soybean, coconut, palm and rape seed plant oil, for example the mixture sold under the reference Akogel® by the company Aarhuskarlshamn (INCI name: Hydrogenated Vegetable Oil),
   butters of plant origin, such as mango butter, such as the product sold under the reference Lipex 203 by the company Aarhuskarlshamn, shea butter, in particular the product whose INCI name is Butyrospermum Parkii Butter, such as the product sold under the reference Sheasoft® by the company Aarhuskarlshamn, cupuacu butter (Rain Forest RF3410 from the company Beraca Sabara), murumuru butter (Rain Forest RF3710 from the company Beraca Sabara), cocoa butter; and also orange wax, for instance the product sold under the reference Orange Peel Wax by the company Koster Keunen,
   and mixtures thereof.

Preferably, the pasty compound is selected from the group consisting of petroleum jelly, esters of a glycerol oligomer, vinyl ester homopolymers containing C₈-C₃₀ alkyl groups, phytosterol esters, fatty acid triglycerides and derivatives thereof, pentaerythritol esters, noncrosslinked polyesters resulting from polycondensation between a linear or branched C₄-C₅₀ dicarboxylic acid or polycarboxylic acid and a C₂-C₅₀ diol or polyol, aliphatic esters of an ester, resulting from the esterification of an aliphatic hydroxycarboxylic acid ester with an aliphatic carboxylic acid, esters of a diol dimer and of a diacid dimer, hydrogenated plant oils, butters of plant origin, or a mixture thereof.

More preferably, among the pasty compounds, petroleum jelly, esters of a glycerol oligomer, butters of plant origin, or a mixture, is preferred.

More particularly, petroleum jelly, diglycerol esters of condensates of adipic acid and of glycerol for which some of the hydroxyl groups of the glycerols have reacted with a mixture of fatty acids such as stearic acid, capric acid, stearic acid and isostearic acid, and 12-hydroxystearic acid, shea butter, or a mixture thereof, will preferably be chosen.

According to a preferred embodiment, the pasty compound is present in an amount ranging from 1% to 50% by weight, preferably from 5% to 30% by weight, relative to the total weight of the composition.

### Galenic Form

The composition of the present invention is suitable to be used as a skin care, make up or cosmetic treatment product. More particularly, the composition of the present invention is in the form of makeup product such as lip balm, lip stick, lip gloss, and so on.

The composition of the present invention is solid. The definition of “solid” is described above.

The composition of the present invention is anhydrous, which is also defined above.

According to a preferred embodiment, the compositions that are more particularly under consideration according to the invention are solid and are in the form of cast products, obtained by hot-casting in a mould, and cooling to room temperature via a phenomenon of setting to a solid or via a cooling tunnel according to the industrially available tools, which are well known to those skilled in the art.

Their texture is thick and is comparable to that of a butter.

The compositions according to the invention in the form of a cast product are endowed with satisfactory properties in terms of sensory, in particular, no oily feeling, and they are such that they give the user a very good spreading during application, and meanwhile does not break when using at a high temperature, such as 37° C. Besides, the cast product of the composition of the present invention has an improved aesthetic appearance after long time storage.

### Additives

In a particular embodiment, a cosmetic composition according to the invention further comprises at least one compound chosen from, hydrophilic solvents, lipophilic solvents and oils different from the nonvolatile oils cited before, and mixtures thereof.

A cosmetic composition according to the invention may also comprise any additive usually used in the field under consideration, chosen, for example, from gums, anionic, cationic, amphoteric or nonionic surfactants, silicone surfactants, resins, thickening agents, fillers, dispersants, antioxidants, essential oils, preserving agents, fragrances, neutralizers, antiseptics, UV-screening agents, cosmetic active agents, such as vitamins, moisturizers, emollients or collagen-protecting agents, colorants, and mixtures thereof.

Accordingly, the composition of the present invention may further comprise at least one filler. Preferably the filler is hydrophobic silica aerogel particle.

It is a matter of routine operations for a person skilled in the art to adjust the nature and amount of the additives present in the compositions in accordance with the invention such that the desired cosmetic properties and stability properties thereof are not thereby affected.

The composition according to the invention may be prepared in a conventional manner.

### Method and Use

According to an embodiment, the present invention relates to a non-therapeutic method for treating a keratin material, comprising the step of applying the composition of the present invention to the keratin material.

Preferably the present invention relates to a method for making up the skin, especially the lips, comprising the step of applying the composition of the present invention to the skin and in particular the lips.

According to another embodiment, the invention relates to the use of a composition of the present invention in making up a keratin material, especially the skin, and more particularly the lips.

The examples that follow are given as nonlimiting illustrations of the present invention. The percentages are weight percentages.

### EXAMPLES

### Formulation Examples

The following formulation examples were prepared.

| | % by active ingredient (wt %) | | | |
|---|---|---|---|---|
| | Invention | | | |
| | formula | Comparative formula | | |
| Ingredient INCI name | 1 | 1 | 2 | 3 |
| Polyethylene (Performalene | 6 | 6 | 6 | 6 |
| 500-L from New Phase | | | | |
| Technologies) | | | | |
| Beeswax (White beeswax SP | 0 | 4.5 | 0 | 0 |
| 453P from Starhl & Pitsch) | | | | |
| Hydrogenated myristyl olive | 0 | 0 | 4.5 | 0 |
| esters (Phytowax ® olive | | | | |
| 14L48 from Sophim) | | | | |
| Cetyl esters (and) cetyl esters | 4.5 | 0 | 0 | 1 |
| (mixture of myristyl stearate and | | | | |
| myristyl palmitate, Paester 9161 | | | | |
| from Patech Fine Chemicals) | | | | |
| Silica silylate (Dow Corning | 0.8 | 0.8 | 0.8 | 0.8 |
| VM-2270 Aerogel Fine | | | | |
| Particles from Dow Corning) | | | | |
| Petrolatum (Ultima white pet | 15 | 15 | 15 | 15 |
| usp from Calumet Specialty) | | | | |
| Bis-diglyceryl polyacyladipate-2 | 7.6 | 7.6 | 7.6 | 7.6 |
| (Softisan ® 649 from Cremer | | | | |
| oleo) | | | | |
| Diisostearyl malate | 17.5 | 17.5 | 17.5 | 17.5 |
| (Schercemol ™ DISM ester | | | | |
| from Lubrizol) | | | | |
| Polybutene (Indopol ® H 1500 | 12 | 12 | 12 | 12 |
| from Ineos) | | | | |
| Polybutene (Indopol ® H 100 | QS to | QS to | QS to | QS to |
| from Ineos) | 100 | 100 | 100 | 100 |
| Octyldodecanol (Eutanol ® G | 16 | 16 | 16 | 16 |
| from BASF) | | | | |
| Shea butter (Sheasoft ® from | 3.0 | 3.0 | 3.0 | 3.0 |
| Aarhuskarlshamn) | | | | |
| Preservatives | 0.05 | 0.05 | 0.05 | 0.05 |
| Pigments | 4.0 | 4.0 | 4.0 | 4.0 |
| Fragrance | 0.03 | 0.03 | 0.03 | 0.03 |

Comparative formula 1 contains a combination of waxes, both of which has a melting point of greater than 60° C.; comparative 2 does not contain wax b) as claimed, comparative formula 3 has a ratio of wax a) and b) as described in the invention greater than 4.

### Protocol of Preparation

The above listed formulations examples are prepared following the steps of:

mixing all the ingredients of the invention example 1 and comparative examples 1-3 respectively, under the temperature 95° C.;

stirring the mixture until homogenization.

### Evaluation Example

Evaluations on the sensory, in particular, the oily feeling, spreading, pay-off, aesthetic appearance after 3 months storage, mechanical property when using at 37° C. were examined.

Mechanical property of the formulas was evaluated using crash test, by following steps:

heat the invention and comparative examples to 37° C. for 24 hours;

apply the example to the lips under the heated temperature.

Sensory was evaluated by 6 panelists, by applying the formulas listed above to the lips.

Spreading and pay-off properties were evaluated by 5 experts by the following steps:

repeatly apply the formulas 3 times on the same area of the forearm using the same force;

weigh the weight loss of the product;

measure the size of the area on the forearm where the formula was applied;

calculate the weight loss per square centimeter.

Aesthetic appearance was evaluated by hot-casting the invention and comparative formulas into a mould, cooling down the formulas to form a solid cast products, and store the products for 3 months at room temperature and atmospheric pressure.

Finally, comments or scores were given by the panelists and experts on the above mentioned properties.

5: very good;

4: basically good;

3: acceptable;

2: slightly poor and not acceptable;

1: poor, not acceptable.

| | Invention | Comparative | Comparative | Comparative |
|---|---|---|---|---|
| Properties | formula 1 | formula 1 | formula 2 | formula 3 |
| Spreading | 5 | 2, too draggy | 5 | 4 |
| Aesthetic | 5 | 5 | 1, white | 5 |
| appearance | | | crystal | |
| after 3 months | | | appear | |
| storage | | | | |
| Sensory (no | 5 | 2, too draggy | 4 | 2, too oily |
| oily feeling) | | | | |

It was observed that all the formulas listed above have good mechanical properties and do not break after 10 strokes at 37° C.

It was also observed that all the formulas have good pay-off on the lips after application.

Based on the above listed evaluation results, the inventors discovered that the invention formula 1 possesses an improved non-oily feelings after application on the lips, an improved spreading, in particular, non-draggy feelings during application, and an improved aesthetic appearance after 3 months storage, comparing to the comparative formulas 1 to 3, respectively.

1. A solid anhydrous composition comprising:
a) at least one wax with a melting point greater than or equal to 60° C.;
b) at least one wax, different from a), of solid linear ester derived from C₆-C₃₀ fatty acid;
c) at least one nonvolatile oil; and
d) at least one pasty compound,
wherein the weight ratio of a) wand h) is from 0.05 to 4.

2. The composition of claim 1, wherein the wax a) is selected from the group consisting of waxes of natural origin, waxes of synthetic origin, or a mixture thereof.

3. The composition of claim 1, wherein the wax a) is present in an amount ranging from 1% to 20%, by weight relative to the weight of the composition.

4. The composition of claim 1, wherein the wax b) is a solid linear ester derived from C₁₀-C₂₂ fatty acid, and comprising from 25 to 36 carbon atoms, or its mixture.

5. The composition of claim 1, wherein the wax b) is selected from the group consisting of myristyl myristate, myristyl palmitate, myristyl stearate, cetyl myristate, cetyl palmitate, cetyl stearate, stearyl myristate, stearyl palmitate, stearyl stearate, or a mixture thereof.

6. The composition of claim 1, wherein the wax b) is present in an amount ranging from 0.5% to 20%, relative to the weight of the composition.

7. The composition of claim 1, wherein the weight ratio of a) and b) is from 0.5 to 2.

8. The composition of claim 1, wherein the nonvolatile oil is selected from the group consisting of nonvolatile hydrocarbon-based oils.

9. The composition of claim 1, wherein the nonvolatile oil is present in an amount ranging from 3% to 80% by weight, relative to the total weight of the composition.

10. The composition of claim 1, wherein the pasty compound is selected from the group consisting of lanolin and derivatives thereof, petroleum jelly, polyol ethers chosen from ethers of pentaerythritol and of polyalkylene glycol, ethers of fatty alcohol and of sugar, and mixtures thereof, polymeric or nonpolymeric silicone compounds, polymeric or nonpolymeric fluoro compounds, vinyl polymers, liposoluble polyethers resulting from the polyetherification between one or more C₂C₁₀₀ diols, esters, and mixtures thereof.

11. The composition of claim 1, wherein the pasty compound is present in an amount ranging from 1% to 50% by weight, relative to the total weight of the composition.

12. The composition of claim 1, wherein the composition is in form of a cast composition.

13. A non-therapeutic method for treating a keratin material, comprising applying a composition of claim 1 on the keratin material.
